(19) United States
(12) Patent Application Publication
(10) Pub. No.: US 2012/0164718 A1
(43) Pub. Date: Jun. 28, 2012

(54) Removable/disposable apparatus for MEMS particle sorting device

(75) Inventors: Jamie H. Bishop, Goleta, CA (US); David M. Erlach, Santa Barbara, CA (US); Ian S. Foster, San Francisco, CA (US); John S. Foster, Santa Barbara, CA (US); John C. Harley, Santa Barbara, CA (US); Douglas L. Thompson, Santa Barbara, CA (US)

(73) Assignee: Innovative Micro Technology, Goleta, CA (US)

(21) Appl. No.: 12/149,637

(22) Filed: May 6, 2008

(51) Int. Cl. C12M 1/34; B23P 11/00; H05K 13/00

(52) U.S. Cl. 435/288.7; 29/592.1; 29/426.1; 29/426.2

(57) ABSTRACT

A micromechanical particle sorting system uses a removable/disposable apparatus which may include a compressible device, a filter apparatus and a cell sorter chip assembly. The chip assembly may include a tubing strain relief manifold and a microfabricated cell sorting chip. The chip assembly may be detachable from the filter apparatus in order to mount the MEMS particle sorting chip adjacent to a force-generating apparatus which resides with the particle sorting system. A disturbance device installed in the particle sorting system may interact with a transducer on the removable/disposable apparatus to reduce clogging of the flow through the system. Using this removable/disposable apparatus, when the sample is changed, the entire apparatus can be thrown away with minimal expense and system down time.

### CROSS REFERENCE TO RELATED APPLICATIONS

This U.S. Patent application is related to U.S. patent application Ser. No. 10/189,607, now U.S. Pat. No. 6,838,056, U.S. patent application Ser. No. 11/196,291, filed Aug. 4, 2005, now U.S. Pat. No. 7,220,594, and to U.S. patent application Ser. No. 11/260,367, now U.S. Pat. No. 7,229,838. Each of these is incorporated herein by reference in its entirety for all purposes.

### STATEMENT REGARDING FEDERALLY SPONSORED RESEARCH

Portions of the present invention were made with U.S. Government support under DARPA Grant No. DAMD 17-02-2-0067. The government may have certain rights in this invention.

### STATEMENT REGARDING MICROFICHE APPENDIX

Not applicable.

### BACKGROUND

1. Field of the Invention

This invention relates to the sorting of particles, such as biological cells. More particularly, this invention relates to a microelectromechanical systems (MEMS) particle sorting apparatus used to sort a component of interest from the rest of a fluid sample.

2. Description of Related Art

Many new therapies for cancer patients relate to enabling them to better withstand the challenge made to their bodies by the chemotherapies. In particular, it has recently been found that the inability of some patients to cope with chemotherapies has to do with the destruction of hematopoietic stem cells (HSCs), as ancillary damage of the chemotherapy. HSCs are the progenitor cells found in bone marrow, peripheral blood and many lymphoid organs. HSCs are responsible for generating the immune system components, such as T-cells, as well as the vital components of blood. When HSCs are destroyed in sufficient numbers, it becomes difficult for patients to replace blood cells, resulting in anemia often suffered by patients. The destruction of HSC's is also a leading cause of death in radiation victims, as the progenitor cells are destroyed, thereby destroying the ability to regenerate the vital components of the blood and immune systems.

Recent research has indicated however that if the human hematopoietic stem cells are removed from the patients' bodies prior to their receiving chemotherapy, and then replaced after the chemotherapy, the human hematopoietic stem cells are shielded from the effects of the chemotherapy. By re-infusing the human hematopoietic stem cells after the chemotherapy is finished, the patients' ability to regenerate their blood cells is regained and their resilience to the therapy is greatly enhanced. As a result, higher dosages of the chemotherapy can be administered to patients with better chances of diminishing the viability of the cancer cells, and yet the patients are able to re-graft their blood-forming HSCs, which have been protected from exposure to the chemotherapy.

Until recently, the standard treatment for patients requiring blood-forming system reconstitution after chemotherapy was a bone marrow transplant (BMT). Bone marrow transplants require up to 100 withdrawals of marrow from the hip bone by large needles and the subsequent re-infusion of large volumes of cells and other fluid. These procedures are highly invasive, cumbersome, expensive and pose additional risks to the patient.

Mobilized peripheral blood (MPB), which accomplishes the same post- chemotherapy reconstitution with less trauma to the donor, can be generated in most patients by injecting a granulocyte colony-stimulating factor (G-CSF) that causes the body to produce a sufficient quantity of hematopoietic stem cells (HSCs). These cells migrate from the bone marrow to the blood, from which they are harvested in a sufficient quantity in a single 2-4 hour session that only requires vein access.

Both the bone marrow extractions and mobilized peripheral blood from cancer patients contain the hematopoietic stem cells necessary for reconstitution; however, they also contain large numbers of cancer cells, which are re-infused into the patient along with the human hematopoietic stem cells after the chemotherapy treatment. Logic and an increasing body of literature suggest that this reintroduction of cancer cells is one cause of the limited survival improvement associated with high dose chemotherapy and cell transplant.

Therefore, technology was developed to obtain highly purified non-cancerous HSCs from mobilized peripheral blood; i.e., the purification process eliminates the cancer cells, but retains the healthy stem cells necessary for reconstitution. The purification process also reduces the transfusion volume to less than 0.1 ml, in contrast to the 500-1500 ml of cells in fluid volume for BMT and MPB. The purification process is performed by flow cytometry, which separates the constituents of a fluid sample mixture according to fluorescence detected from the constituents. Purity of the resulting HSC product was 95% by this method, with no detectable cancer cells, and further details of the methodology can be found in Negrin et al., “Transplantation of Highly Purified CD34⁺Thy-1⁺Hematopoietic Stem Cells in Patients with Metastatic Breast Cancer”, Biology of Blood and Marrow Transplantation 6:262-271 (2000). For patients undergoing this HSC reinfusion treatment, the 5-year survival rate for women with advanced metastatic breast cancer jumped from 5% to about 50%.

Another application for HSC sorting is protection against nuclear radiation effects. The procedure would be to sort HSCs from individuals who potentially could be exposed at some later date to nuclear radiation. The human hematopoietic stem cells are frozen and can survive in that state essentially forever. If the individual is exposed, as could be the case in a nuclear plant accident or warfare, the human hematopoietic stem cells are then shipped to the patient's location, rapidly thawed, and then re-inserted into the patient. This procedure has been shown to save animals exposed to otherwise lethal doses of radiation.

More recently, other populations blood stem cells and/or progenitor or moderately differentiated stem cells have been shown to be clinically efficacious. Such populations have shown promise in providing effective treatment for a variety of serious or lethal illnesses. These illnesses may include circulatory disorders such as chronic heart failure and critical limb ischemia, and childhood metabolic disorders such as Tay-Sachs and Krabbe diseases. These afflictions may be treated by injecting populations of HSC and progenitor cells, which then set about to repair the damaged tissues. Indeed, the repair of circulatory damage may become the primary application for concentrated, purified populations of stem and progenitor blood cells.

However for these treatments to become practical, it must be learned how to sort large quantities of viable hematopoietic stem cells from the other constituents of the blood, with high concentration and high purity. An estimate of the number of stem cells required is 4×10⁶ stem cells/kg body weight. The present separation process, flow cytometry, uses a high-pressure nozzle to separate tiny droplets containing the cells. The cell suspension is brought to the nozzle assembly under positive pressure, and introduced to the center of the sheath flow. The properties of fluid laminar flow focus the cell suspension into a single file, which is confined to the center of the fluid jet. Droplets are formed as the fluid exits the nozzle, and the droplets pass through one or more laser beams, which irradiate the cells and excite fluorescent markers with which the cells are tagged. The droplets are then given an electric charge to separate the droplets containing HSCs from those containing other constituents of the blood, as detected by fluorescence of the tagged molecules. The droplets are separated by passing them between a pair of electrostatic plate capacitors, which deflect the charged droplets into a sorting receptacle. The time-of-flight of the droplet through these stages requires careful calibration so that the sorting efficiency and effectiveness can be optimized.

Among the difficulties with the process is speed, as throughputs are limited to about 40,000 events per second. The rate is limited by the amount of pressure that the cells can withstand without damaging their viability, and the flow rate is proportional to the pressure. The fluidic settings which control the conditions of operation of the flow cytometers are interrelated. The nozzle diameter, system pressure and droplet frequency are independently set, whereas the jet velocity is related to the system pressure and nozzle diameter. Therefore the droplet time-of-flight must be set by empirical calibration with a standard sample. Therefore, not only are the systems themselves quite expensive, they require trained engineering staff to operate effectively. And lastly, contamination of the vessels with old sample tissue is a problem, as the equipment is difficult to sterilize. Decontamination issues encourage the use of disposable vessels, for which these machines are presently not designed. The high pressures used in the machines favor permanent fixturing of the plumbing in the tools. Also the careful alignment required of the receptacles with the trajectories of the droplets favors the permanent installation of the receptacles. About 7000 such systems exist worldwide today, and tend to be research tools rather than production equipment which can be used for clinical sorting in treating patients.

### SUMMARY

Therefore, a need exists for a separation technique that solves throughput, cost, and disposability issues associated with present methods. This disclosure describes a novel device and method based on microelectromechanical systems (MEMS). MEMS devices are micron-sized structures which are microfabricated using photolithographic techniques pioneered in the semiconductor processing industry. Due to their small size and the batch fabrication techniques used to make the structures, they are capable of massive parallelism required for high throughput. These same features make them relatively inexpensive to fabricate, so that a disposable system is a realistic target for design.

A microfabricated cell sorting system is described in U.S. Pat. No. 6,838,056 (Attorney Docket No. IMT- CellSorter), incorporated by reference in its entirety. The system uses a microfabricated MEMS chip to sort a component of interest from the remainder of a fluid sample stream. Important details of such a MEMS-based particle sorting system are described in related U.S. Pat. Nos. 7,220,594 (Attorney Docket No. IMT- CellSorterOptics), and U.S. Pat. No. 7,229,838 (Attorney Docket No. IMT-CellSorterMotor), incorporated by reference herein in their entireties. This disclosure relates to a removable and/or disposable apparatus usable in the aforementioned cell sorting system. All the components of the removable/disposable apparatus may be detached from the cell sorting system and cleaned, replaced or disposed of, when a sample changes or a component needs to be replaced. Accordingly, all components are designed to be inexpensive and/or sterilizable.

The removable/disposable apparatus may include the microfabricated particle sorting chip held securely in a fixture, referred to herein as a chip assembly, which may include a strain relief manifold which may hold the flexible tubes leading to and from the microfabricated particle sorting chip. The flexible tubes may include an input tube which delivers the fluid sample from one or more flexible sample bags to the microfabricated particle sorting chip, and two output tubes, one for the unwanted (waste) particles and another for the wanted (sorted) particles. During operation of the system, the flexible sample bags may be held in a pressure chamber with less than about 2.0 atm pressure, which forces the flow of the fluids out of the one or more sample bags and through the microfabricated particle sorting chip at a well-defined fluid flow rate of between about 10 and about 75 milliliters per hour.

The removable/disposable apparatus may also include a filter for filtering larger particles and debris from the input sample delivered from the one or more flexible sample bags. The filter may include a polyethersulfone (PES) membrane with about 15 μm holes, which removes larger particles from the sample stream. The filter may prevent the clogging of the microfabricated particle sorting chip by these larger particles. The filter may be installed in a filter carrier and detachably mounted on the microfabricated particle sorting system for operation.

The chip assembly with the microfabricated particle sorting chip may be clamped to the filter carrier for transport and installation in the cell sorter system. During installation of the removable/disposable unit, the chip assembly may be disengaged from the filter carrier, in order to reposition the microfabricated particle sorting chip in the proper orientation for interaction with a distinguishing means and a force-generating apparatus, as described fully in the incorporated '594 and '838 patents. The distinguishing means may identify the component of interest from the remainder of the fluid stream. The force generating apparatus may activate the microactuators built on the microfabricated cell sorting chip to direct the component of interest to a special sort receptacle, when triggered to do so by the distinguishing means. Alternatively, the microfabricated cell sorting chip may use other, non-mechanical means to separate the component of interest from the fluid stream, such as differential pressure or differential flow, electric or magnetic fields, for example. However, since the distinguishing means and force-generating apparatus may be relatively large and complex systems, they may reside permanently within the cell sorting system rather than being a part of the microfabricated particle sorting chip and removable/disposable apparatus.

The removable/disposable apparatus may also include a compressible device such as a rubber bung which seals the pressure chamber in which the sample bags are held, and allows passage of the sample tube lines leading from the sample bags to the filter. Tubes can be molded directly into the rubber of the bung, so that no breach in the tubing is necessary in order to pass through the wall of the pressure chamber.

After the fluid stream passes through the microfabricated particle sorting chip, the sorted particles are directed into a sort stream and sort receptacle, whereas the unwanted particles are delivered to a waste stream and waste receptacle.

If the sample needs to be replaced with a new sample, for example from another patient, the entire removable/disposable apparatus may be easily disengaged from the particle sorting system and thrown away. It may then be replaced with another removable/disposable apparatus, all of the constituents of which are sterile, and remounted in the particle sorting system. Alternatively, if only the microfabricated particle sorting chip needs to be replaced, for example in the event of clogging, it can easily be removed from the chip assembly, replaced with a new microfabricated particle sorting chip, and replaced in the machine.

Since none of the laser system, pumping system, force-generating apparatus or any other parts of the particle sorting system need to be replaced when the samples are changed, this approach leads to substantial cost savings in the operation of the microfabricated particle sorting device. Since all of the components of the removable/disposable apparatus are sterile or sterilizable, improved cleanliness and reduced likelihood of sample contamination are achievable using the removable/disposable apparatus described here.

Accordingly, the removable/disposable apparatus may include a sample holder which holds at least one component of a sample stream, a filter which receives the sample stream from the sample holder and filters particles from the sample stream, and is configured to be repeatably coupled to and decoupled from the particle sorting system, first flexible tubing which delivers the sample stream from the sample holder to the filter carrier, a compressible device disposed in or around the first flexible tubing, and a chip assembly holding a microfabricated particle sorting chip which receives the sample stream from the filter.

These and other features and advantages are described in, or are apparent from, the following detailed description.

### BRIEF DESCRIPTION OF THE DRAWINGS

The present invention will be understood more fully from the following detailed description, and from the accompanying drawings, which however, should not be taken to limit the invention to the specific embodiments shown but are for explanation and understanding only. In the figures, like numbers may refer to the same, or analogous features in the various views.

FIG. 1 is a simplified view of the components of the removable/disposable apparatus for the particle sorting system;

FIG. 2 is a simplified view of the components of the removable/disposable apparatus for the particle sorting system in greater detail, and showing the pressure chamber;

FIG. 3 is a simplified view of the sample and buffer bags in the pressure chamber, along with ancillary control equipment;

FIG. 4 is a simplified view of the filter carrier of the removable/disposable apparatus for the particle sorting system;

FIG. 5 is a simplified view of the chip holder assembly of the removable/disposable apparatus for the particle sorting system; and

FIG. 6 is a simplified view of the removable/disposable apparatus installed in the particle sorting system.

### DETAILED DESCRIPTION

The systems and methods set forth herein are described with respect to a particular embodiment, that of a cell sorter for sorting certain cells, such as human hematopoietic stem cells from a sample containing other cells or whole blood. However, it should be understood this embodiment is exemplary only, and that the systems and methods may be applicable to a wide range of sorting applications, wherein it is desired to separate a particular component of interest from a remainder of a fluid stream. Thus, while the terms “MEMS cell sorting device” may be used herein, it should be understood that the systems and methods described here may be applicable to any situation in which small particles need to be separated from a sample stream, not just biological cells. The particle sorting device may also use non-mechanical separation means, such as pressure differentials, electric or magnetic fields to separate the particles in a microfluidic device.

The systems and methods described herein are directed to the disposable components of such a particle sorting system. Some details of an exemplary particle sorting system in general and microfabricated cell sorting chip in particular are described first, followed by details of the removable/disposable apparatus used in the particle sorting system.

The particle sorting system may be a MEMS cell sorting system, and may thus include a MEMS, or microfabricated cell sorting chip. The MEMS cell sorting chip may include an array of parallel inlet channels fabricated in a wafer, with each channel having a characteristic dimension of about 25 μm just large enough to allow the passage of a hematopoietic stem cell (HSC), for example. In another embodiment, the microfabricated channels may be roughly square in cross section, with a characteristic dimension of about 30 μm. Hematopoietic stem cells are typically between 5 and 10 um in diameter. At the exit from each parallel channel may be an independent valve/actuator. The actuator may direct the cells individually into one of two or more different possible pathways, which are microfluidic channels etched into the wafer, beneath the parallel channels. The actuator may be directed to move upon distinguishing the particles of interest, for example, HSCs, from the sample stream by a distinguishing means. The distinguishing means may generate a signal indicating that the target particle is in a position to be sorted, at which point a signal may be generated for a separation means, such as a microfabricated actuator. The actuator may be caused to move by the application of force by a force-generating apparatus located within the cell sorting system, as further described below.

The particle sorting system may thus include a means of distinguishing a particle of interest from a fluid stream, along with a separation means which directs the particle of interest in one of a plurality of exit paths within the particle sorting system. The means for distinguishing may be a laser irradiation source in which laser light is directed to appropriately tagged particles, which emit a fluorescent signal in response to the irradiation. The emitted signal is detected by an optical detector, and the signal from the optical detector is fed to a controlling computer or microprocessor. The microfabricated cell sorting chip may also include an optically transparent layer which has reflective and refractive optical elements formed therein, which serve to focus the excitation laser to a point just before the particle encounters the microfabricated actuators. The laser irradiation of the sample stream may cause appropriately tagged particles to fluoresce, and the fluorescence signal may be detected by the optical detector. Additional details as to the design and manufacture of these optical elements may be found in the incorporated '594 patent.

Upon receiving an indication that a component of interest has been identified in the sample stream, the computer or microprocessor may then direct a separating means to separate the target particle from the remainder of the sample stream In one embodiment, the separation means includes a force-generating apparatus which moves a microactuator in order to direct the particle of interest into the appropriate exit path, either as a sorted (saved) particle or as an unwanted (waste) particle. The force-generating apparatus may be electromagnetic, i.e. a magnetizable member or core around which is wound at least one turn of a current-carrying conductor. The magnetizable member or core then produces a magnetic flux which may interact with a magnetizable portion affixed to an actuator in the microfabricated cell sorting chip. Alternatively, the force-generating apparatus may produce an electric field which may interact electrostatically with another conductive surface to pull an actuator in or push an actuator out. The force-generating apparatus may thereby operate the microfabricated actuator to direct each of the components of the fluid stream into a separate storage receptacle, appropriately labeled either “sort” or “waste”, for example. Additional details as to the construction of the particle sorting system may be found in the incorporated '056 patent. Additional details as to the design and manufacture of the laser distinguishing means, MEMS actuator and force-generating apparatus may be found in the incorporated '594 and '838 patents.

The force-generating apparatus and laser distinguishing means may reside in the particle sorting system, rather than in the MEMS chip itself, in order to reduce the cost of the MEMS cell sorting chip. Since the MEMS cell sorting chip will necessarily come into contact with the sample fluid, it may form a part of the removable/disposable apparatus, and thus it is important to minimize the expense of this part, in order to minimize the cost of the removable/diposable apparatus and the expense of operating the device. In addition, reducing the functionality of the MEMS cell sorting chip limits the number of components that require sterilization, and the materials used for the disposable apparatus are all resilient enough to withstand the sterilization procedure.

Accordingly, the overall particle sorting system may include a removable/disposable apparatus with cell sorting chip, a laser source, a force-generating apparatus, power supplies, a controlling computer. The overall particle sorting system may also include a pressure chamber, which provides the pressure which forces the fluid sample through the rest of the system, as described further below. The components of the cell sorting system apart from the removable/disposable apparatus are generally non-disposable, but are re-used from patient-to-patient and run-to-run. However, since none of these components actually come into contact with the sample cells, there is little or no requirement for sterility of these components.

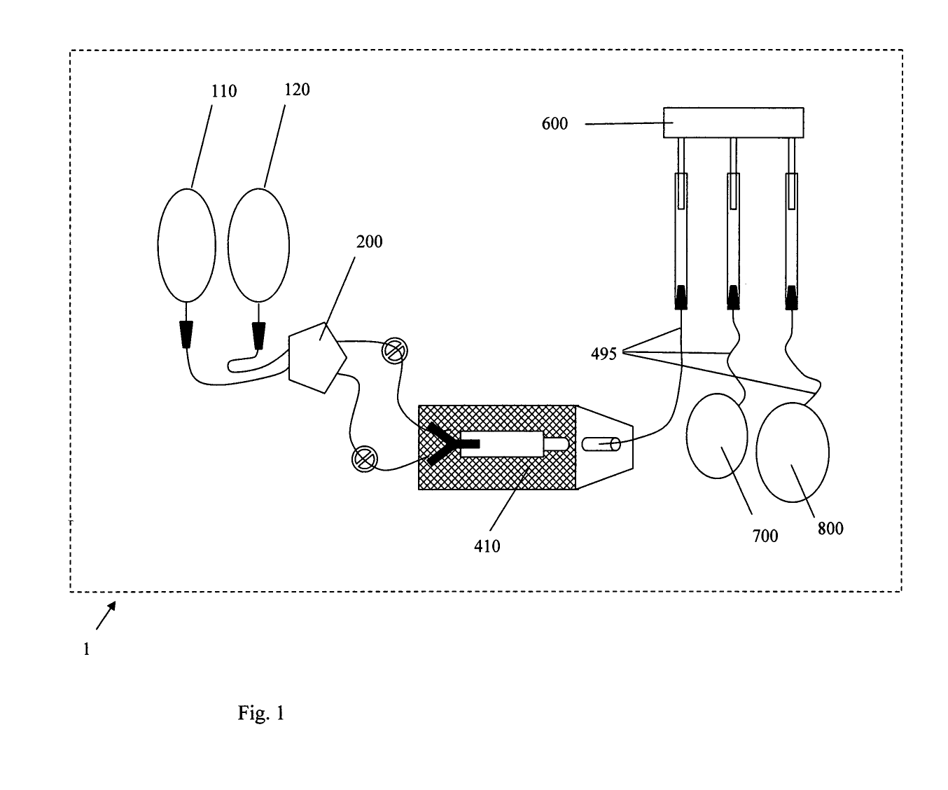

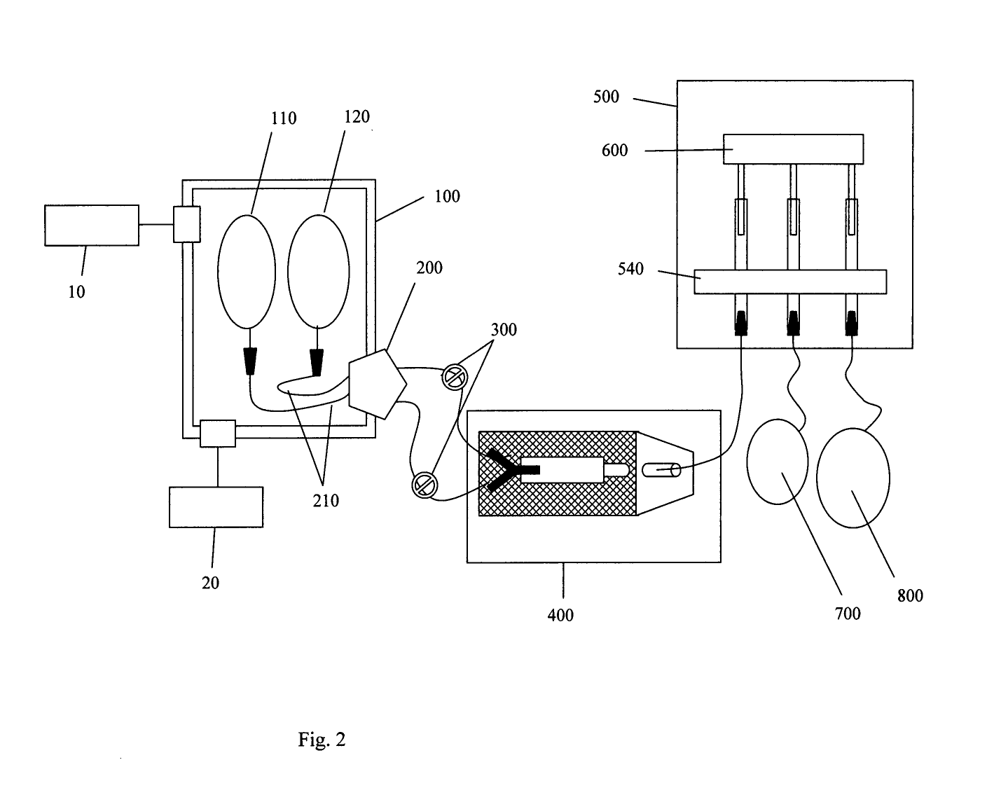

The removable/disposable apparatus is shown in FIG. 1. The removable/disposable apparatus handles the storage and flow of the sample cells and buffer fluid through the cell sorting system. The removable/disposable apparatus 1 includes sample bags, filter, a compressible device, a cell sorter chip, associated tubing and downstream receptacles. These components may be required to be sterile, and are thus disposed of when a new sample is input to the cell sorting system. The components of the removable/disposable apparatus 1 are described first in general with respect to FIG. 1, and then additional details of a preferred embodiment are given with respect to FIGS. 2-6.

The target sample cells may be suspended in a buffer fluid prior to sorting. The buffer fluid may be any convenient medium which can maintain viability of the sample cells, such as phosphate-buffered saline, containing 0.1% to 0.5% fetal calf serum. The cells may have been subjected to pre-treatment, such as removal of cells by filtering, centrifugation, affinity separation or other technique which provides enrichment of the population of cells of interest. In addition, the cells may be diluted with additional fluid to avoid cells being concentrated too close to each other. The fluid mixture is then introduced to the MEMS cell sorting chip under positive pressure, through a filter disposed upstream of the MEMS cell sorting chip. This reduces the tendency of the MEMS cell sorting chip to become clogged.

The sample cells may therefore be stored in a sample bag 110 and a buffer fluid may be stored in a buffer bag 120. Alternatively, these components may be stored in a pre-mixed form, and thus only a single sample bag could be used. From these storage bags, the fluids may be forced through tubing which passes through a compressible device 200 and to a “Y” connector and then through a filter 410. If only a single sample bag is used, only a single line of tubing may be needed and the Y connector may be omitted. From the filter 410, the sample fluid may be transported to the cell sorter chip 600. The cell sorter chip 600 separates the target cells from the buffer fluid and directs them to a sort bag 700, while the unwanted components are directed to a waste bag 800.

The removable/disposable apparatus is shown interfacing with some additional components of the cell sorter system in FIG. 2. As mentioned previously, the sample stream may be introduced to the cell sorter chip from a pressurized chamber 100 containing a sample bag 110 and a buffer bag 120. Pressure in the chamber 100 exerts a pressure on the flexible bags, forcing the fluids out of their respective bags and through the tubes 210. Pressure in the pressure chamber is maintained by the presence of a compressible device 200 disposed in or around the tubing 210. The compressible device may be situated in the wall of the pressure chamber 100. The compressible device may be a compressible stent-like device such as a hose-barb union installed within the tubing which may expand the diameter of the tubing at the location of the stent.

Alternatively, the compressible device may be a deformable plug or bung 200 disposed around the tubing. The durometer of the bung may be about 40 on an A scale, or more generally about 20 to about 60, and may be made of any suitable deformable material such as rubber. In this embodiment, the compressible device may be molded around the tubing to form the deformable bung around the tubing.

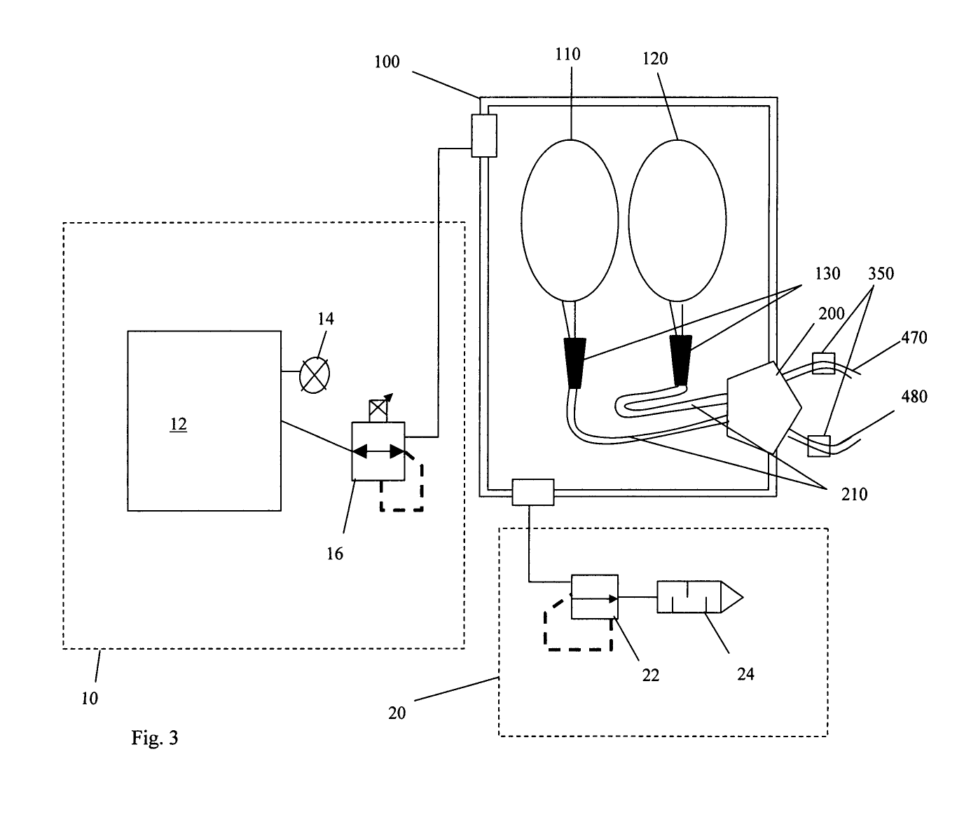

Upon closing the door of the pressure chamber, the compressible device is compressed by the walls of the door which squeeze the compressible device. The compressible device thereby forms a seal around the tubes 210 and prevents the pressure in the pressure chamber 100 from escaping into the environment. From the bung 200, the two lines 210 from the sample bag 110 and buffer bag 120 may pass through pinch valves 300, which can discontinue the flow as desired, to stop the cell sorting process or to replace one or more components. The pinch valves 300 may be manually activated or may be under computer control. Pressure in the pressure chamber 100 may be maintained by a gas supply 10 and pressure limiter 20, and may be set to provide up to about 2 atm pressure. This pressure may result in a flow of about 10 to about 75 milliliters per hour through the cell sorting system. An exemplary embodiment of the pressurized chamber 100 is illustrated in FIG. 3 and described below with reference to that figure.

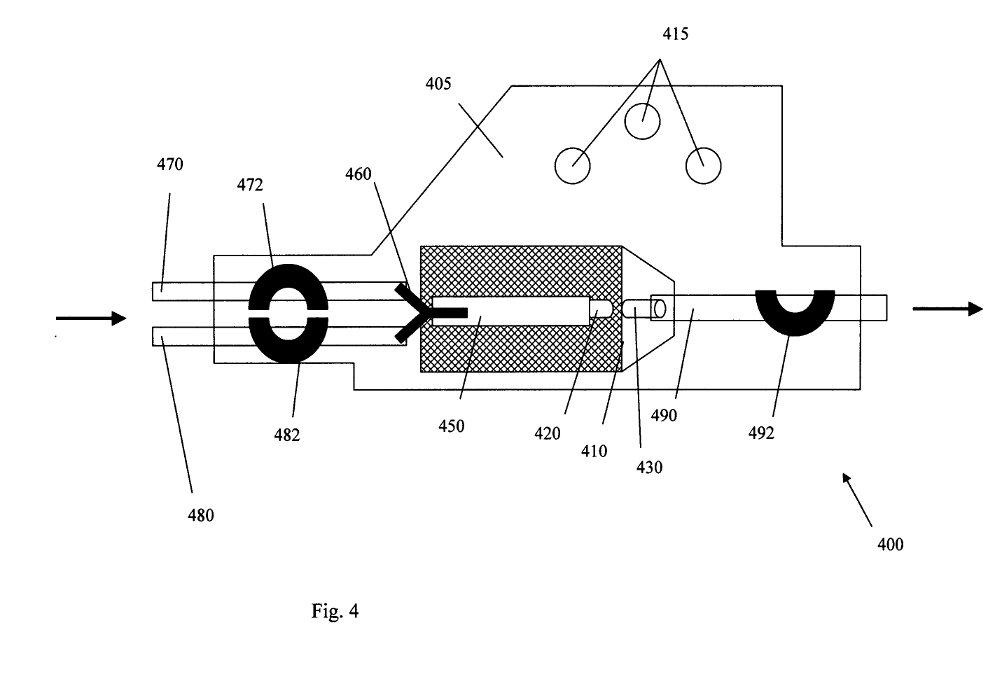

Also as shown in FIG. 2, the filter 410 may be clamped into a filter carrier 400, which in turn may be mounted in the cell sorter system. The filter carrier may also include various tubing clamps and restraint devices that hold the tubes leading to and from the filter 410 in a specific orientation. This may assist the installation of the filter 410 and tubing without tangling of the tubing or inadvertent disconnection of the tubing during installation. An exemplary embodiment of the filter carrier is illustrated in FIG. 4 and described below with reference to that figure.

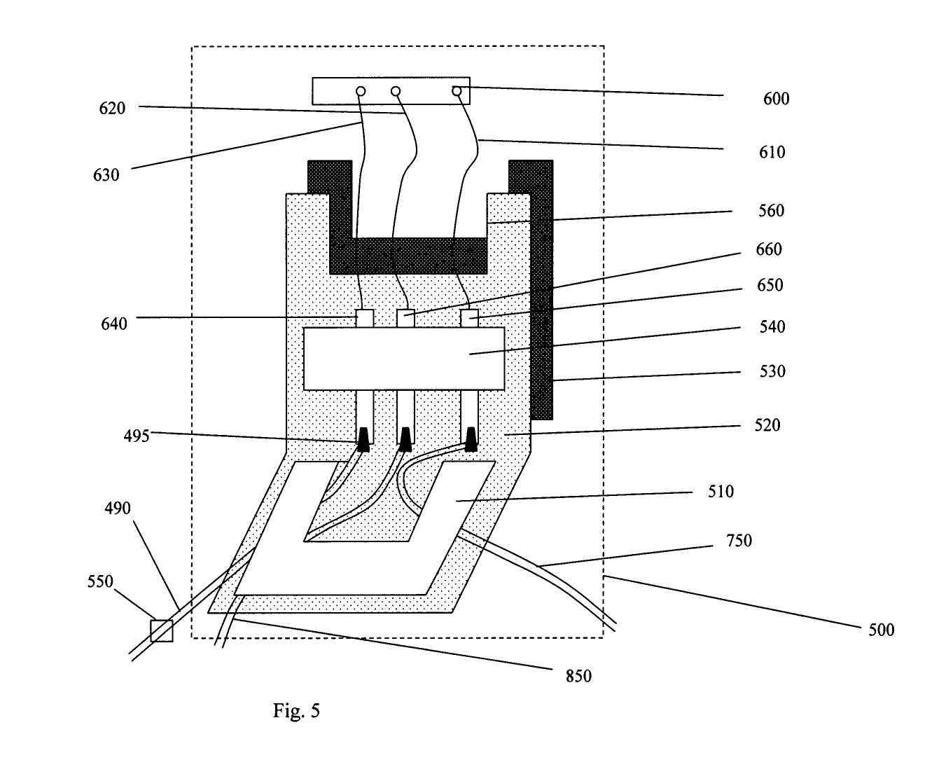

Finally, as also shown in FIG. 2, the MEMS chip 600 may be securely held in a chip assembly 500, which may include a strain relief manifold 540. The strain relief manifold 540 holds the tubes leading to or from the chip in a secure orientation, so that especially the delicate capillary tubes attached to the MEMS chip 600 to not experience excessive strain and resultant breakage. An exemplary embodiment of the chip assembly is illustrated in FIG. 5 and described below with reference to that figure.

Pressure in the pressure chamber 100 may be calibrated and regulated by the apparatus shown in FIG. 3. FIG. 3 shows a gas supply 10 which provides the gas input to the pressure chamber at a pressure determined by a pressure sensor 14 and a regulator 16. The pressure chamber 100 is kept at or below any dangerous limits by a pressure limiter 20, which may include overpressure relief valve 22 and a silencer 24. The combination of the gas supply 10 and pressure limiter 20 keeps the pressure in the pressure chamber at the desired level, and thus the flow of the sample and buffer fluids to the cell sorter at a constant rate 10 to 75 milliliters per hour. Such components are well known in the art and commercially available from a number of sources, and are not described in further detail.

One issue which typically afflicts microfluidic devices such as MEMS cell sorting chip 600 is clogging due to debris, clotting agents or to other constituents of the sample stream. One of the advantages of the pneumatic pressure driven system illustrated in FIG. 3 is that the flow through each microfluidic passage in the MEMS chip 600 remains the same, even if some passages become clogged. Thus, the timing of the actuation of the MEMS actuators based on the signal from the distinguishing means does not need to be adjusted in the event of clogging. This is in contrast to other methods of pumping such as volumetric displacement using, for example, a syringe or plunger. In this case, a certain volume of fluid must be transmitted through the device, so that if some channels become clogged, the flow rate through the remaining open channels is increased. This would then require adjustment of the timing of mechanisms in the cell sorter system.

Similarly, other pumping mechanisms, such as peristaltic pumping, wherein the flexible tubing is deformed or massaged to encourage the fluid flow through the tube, also result in non-uniform flow rates. As the tubes are massaged, a variable, though perhaps regularly varying, flow rate occurs through the tubes. The pneumatic pumping enabled by the pressure chamber 100 enclosing flexible bags 110 and 120 may therefore be superior to other approaches as it results in a steady, uniform flow through the cell sorting system 1000. Using the deformable bung 200 allows the pressure chamber approach to be implemented in a way that still allows easy sterilization and disposability of the removable/disposable apparatus 1.

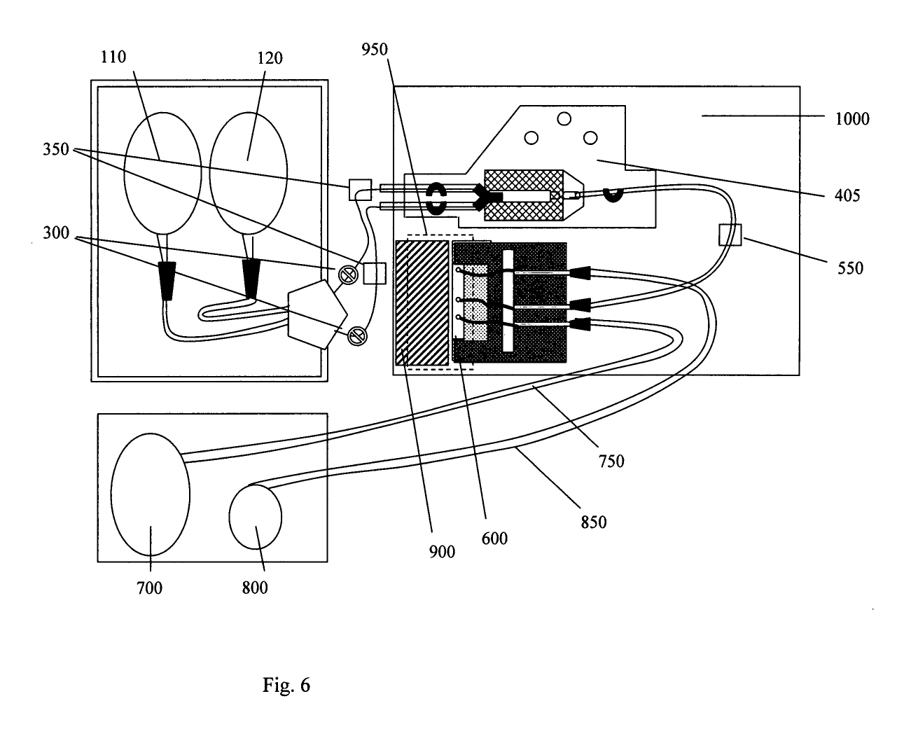

In order to further reduce issues related to clogging of the path of the fluid stream, a disturbance device 350 or 550 may be installed in the cell sorting system 1000. Disturbance device 350 or 550 may be configured to briefly disturb the fluid flow in the fluid path. The duration of the disturbance may be short compared to the time it takes to for an element of the flow to pass from the distinguishing means to the separation means. This disturbance device 350 or 550 may interact directly with one or more of the components along the fluid path, or may interact with a transducer or other mechanism coupled to the elements along the fluid path. These elements may include the flexible tubing 470 and 480 before the filter 410, the flexible tubing 490 before the input to the MEMS cell sorting chip 600, the filter itself 410, or the MEMS cell sorting chip 600, as shown in FIGS. 5 and 6. Disturbance device 350 may interact with tubing 470 and 480 and disturbance device 550 may interact with tubing 490, for example. This disturbance device 350 or 550 may deliver electrical, mechanical or acoustic disturbances such as vibrations to at least one transducer on any of the flexible tubing and/or to the filter 410 and/or to the MEMS cell sorter chip 600. The transducer or mechanism may be, for example, a piezoelectric or electromagnetic device which converts an electrical signal into an audio disturbance, or it may be a membrane that converts an audio disturbance into a mechanical disturbance. Alternatively, the disturbance device 350 or 550 may deliver the disturbances directly to any or all of these components. The disturbances may be transmitted by either directly contacting the transducer or component, or by generating electrical signals or sound waves which may be received by the transducer or components. The disturbance device 350 or 550 may be, for example, a mechanical member attached to a cam on a motor which periodically taps on the component, or an audio sound generator. These disturbances tend to loosen or agitate clumps of material, which can then proceed with the fluid flow through the element.

In order to reduce the tendency of the disturbance device 350, 550 to compromise the fluid seal between these components, the disturbance may be a sudden negative pressure gradient, which smoothly returns the pressure to its normal level. These pressure gradients may occur on a timetable far too short to affect the volumetric flow through the system, and thus the timing requirements described above with respect to the pumping schemes may not be affected. For example, the pressure gradient may be a sudden lowering of the pressure by about 20% over a timetable of about 10 μsec, followed by a return to the nominal pressure over about 100 μsec. However, the pressure gradients may be sufficient to inhibit the coagulation or clumping of the particles in the fluid stream, or may serve to break up such clots upon formation.

Now turning to FIG. 4, additional detail of the filter carrier 400 of the removable/disposable apparatus 1 is shown. As shown in FIG. 4, the filter 410 may be clamped or glued on a filter carrier 400 which, in turn, may be detachably attached to the chassis of the MEMS cell sorter system. In the embodiment shown in FIG. 4, the filter carrier 400 may be clipped to the cell sorter system by three pins which protrude from the chassis of the MEMS cell sorter system through holes 415 in the filter carrier 400. The attachment means may allow repeatable coupling and decoupling of the filter carrier 400 to the cell sorting system 1000, as the removable/disposable apparatus 1 is replaced. The filter carrier 400 may be made using any convenient, rigid material such as plastic or aluminum. As it does not contact the sample directly, it need not be sterilized or sterilizable.

A fluid line 470 from the sample bag 110, having traversed the bung 200 enters the filter carrier 400. The fluid line 470 may go beneath a tubing brace 472 and then enter the Y connector 460. Within the Y connector 460, the fluid stream is combined with the fluid from the buffer line 480 which brings fluid from the buffer bag 120 which has also passed beneath a tubing brace 482. After being combined at the Y connector, the fluid stream which now contains the sample cells as well as the buffer fluid, is directed through the filter 410. The filter 410 may be a polyethersulfone (PES) membrane with 15 μm holes, which rejects particles larger than this pore size from the fluid stream, while allowing the 10 μm HSC cells to pass. Of course, this filter is exemplary only, and other filters with other filter meshes may be chosen depending on the application and the size of the particles expected. More generally, the filter mesh may be smaller than about 100 μm, to reject particles larger than this size from the sample stream. The presence of the filter 410 may therefore reduce the tendency of the cell sorter chip to become clogged with larger-sized debris.

The input orifice 420 and output orifice 430 of the filter 410 may have a different diameter than the other tubing, such that an adapters 450 and 490 may be required to match the diameter of the input orifice 420 and output orifice 430 of the filter 410.

An important aspect of the removable/disposable apparatus 1 is the detachable chip assembly 500. The detachable chip assembly is shown in greater detail in FIG. 5. The detachable chip assembly 500 is designed to be detachably attached to either the filter carrier 400 or a receptacle in the cell sorting system 1000. The detachable chip assembly 500 may be attached to the filter carrier 400 during installation of the removable/disposable apparatus 1 in the cell sorting system, but is then moved to a location adjacent the force-generating apparatus 900 in the cell sorting system 1000 for cell sorting. Thus, the chip assembly 500 is designed to be repeatably attachable to and detachable from, a predefined location within the cell sorting system 1000. An exemplary location of the chip assembly 500 in the cell sorting system 1000 is shown in FIG. 6.

The chip assembly 500 when mounted in the cell sorting system 1000 locates the MEMS cell sorting chip 600 in a particular orientation relative to a force-generating apparatus 900, which, as mentioned previously, resides in the cell sorting system 1000. In the embodiment described here, the force-generating apparatus may be a magnetizable core wound with at least one turn of conductive wire through which current is driven. The current creates a magnetic field which is amplified by the core. The magnetic field in the core traverses a gap between the arms of the core, and therefore exists in the space between the arms of the core. In this space, the magnetic field may interact with a magnetizable portion of the actuator fabricated in the MEMS cell sorting chip 600, which may cause a movement in the actuator toward the core. In this embodiment, it is important that the MEMS cell sorting chip 600 stably abut the force-generating apparatus 900, in order for that interaction to be strong enough to drive the actuator with the required speed and precision.

The movement of the actuator may alter the position of a diverter carried by the actuator, which forces the flow of the particle into a particular one of a plurality of exit pathways. One of these pathways is the sort output line 750 and the other is the waste output line 850. These lines 750 and 850 lead directly to the sort output bag 700 and the waste output bag 800, respectively. The sort output bag 700 and waste output bag 800, as well as the sample bag 110 and buffer bag 120, may be sterilized 100-300 ml blood bags from Terumo Medical Corporation of Somerset, N.J., for example.

The detachable chip assembly 500 may include a tubing brace 510, which provides a secure location for the input and output tubes 490, 750 and 850. The tubing brace 510 may be attached to the chip holder 520 by any convenient means, such as rivets, or adhesive. From the tubing brace 510, the input line 490 and output lines 750 and 850 may go through a reducer 495 before entering adapter tubing 640, 650 and 660, respectively. The strain relief manifold 540 may then hold the adapter tubing 640, 650 and 660 in a stable, predetermined position relative to the MEMS cell sorting chip 600. The strain relief manifold 540 may be glued or screwed, for example, to the chip holder 520. The chip holder 520 may be screwed, riveted, glued or sandwiched between a top (not shown) and bottom piece 530 of a chip carrier. The carrier 530 may be plastic, for example, and stamped or molded, and is the portion which may be handled upon installation in the cell sorting system 1000, as described below. The tubing held in place by the strain relief manifold 540 may be adapter tubing such as Tygon 1/16″ to 0.03″ which adapts the relatively large gauge flow tubing to the very small gauge capillary plumbing tubes, 610, 620 and 630. The capillary tubing may typically be made of polyimide jacketed quartz or a polymer material such as polyetheretherketone (PEEK) which may be 255 μm×510 μm. These fine tubes may, in turn, be glued to the orifices of the MEMS chip using, for example, a two-part 5-minute epoxy, or any of a number of suitable medical grade adhesives. The narrow gauge PEEK tubing to/from the MEMS cell sorter chip may be for example, about 3 cm to about 6 cm long, whereas the larger gauge flow tubing may be about 20-30 cm long.

The chip holder 520 may also include a nest site 560 which accepts the MEMS cell sorter chip 600. The nest site 560 may be formed by wire EDM for example, to precise specification, so that the MEMS cell sorter chip 600 fits snugly into the nest site 560. The MEMS cell sorter chip 600 may be glued into a stable position using an epoxy, for example.

The removable/disposable apparatus may be assembled by hand or by automated machinery in a factory setting. The MEMS cell sorter chip 600 may be fabricated using the systems and methods set forth in the incorporated '838 and '594 patents. The capillary tubes 610, 620 and 630 may then be glued to the MEMS cell sorter chip 600 using, as mentioned, a two-part 5-minute epoxy, or other suitable medical grade adhesive. The larger gauge tubing may be connected to the smaller gauge capillary tubing using a UV-curable epoxy, using an overlap between the tubes of at least about the width of the larger tube. Alternatively, a sterile tube welder may be used to weld the tubes. The larger gauge tubing 490 may then be connected to the filter 410. This assembly may then be tested under pressure before attachment of the sample and buffer bags to the filter input port 420, to assure that no leaks are present.

To attach the sample and buffer bags 110 and 120, input tubes 210 from the sample and buffer bags may be slipped through corresponding openings in the bung 200. Alternatively, the bung 200 may be molded around the tubes 210. Now referred to as lines 470 and 480 upon exiting the bung, lines 470 and 480 may then be fit over the Y-connector ports 460. The output tube 450 from Y-connector 460 to the filter input 420 may then be attached. These attachments may be simply slip fit, tube-welded or glued with UV epoxy, for example. The entire removable/disposable apparatus 1 may then be again checked for leaks.

When installing the removable/diposable apparatus 1 in a cell sorter system 1000, the filter carrier 400 may first be clamped to the chassis of the cell sorter system 1000, using pins and corresponding openings 415 located on the filter carrier 400 as was illustrated in FIG. 4. The sample and buffer bags 110 and 120 may then be placed in the pressure chamber 100. The bung 200 is then installed in a corresponding receptacle in a wall of the pressure chamber 100, and the pressure chamber door may be closed over the bung 200.

The lines 470 and 480 exiting the bung may be threaded through the pinch valves 300. Finally, the detachable chip assembly 500 may be detached from the filter carrier 400 and placed against the force-generating apparatus 900 in the cell sorter system 1000. The MEMS cell sorter chip 600 may need to be at a well defined and stable abutment to the force-generating apparatus, in order to achieve efficient functioning of the device with high throughput and sort purity. The output lines 750 and 850 from the MEMS cell sorter chip lead to the sort and waste receptacles 700 and 800, which may be stored in any convenient location near or in the cell sorter system 1000. Pressure may then be applied to the flexible bags in the pressure chamber, starting the flow of fluid through the cell sorter system, and the sorting operation may commence.

Upon installation in the cell sorting system 1000, the disturbance devices 350 and/or 550 may be coupled to the desired component of the removable/disposable apparatus 1. This may involve threading the appropriate flexible tubing into an engagement position with the disturbance device, or coupling the disturbance device to a transducer mounted on a component of the removable/disposable apparatus 1.

As the sample fluid passes from the filter 410 and through the MEMS cell sorting chip 600, it may pass by the distinguishing means, shown schematically as reference number 950 in FIG. 6. The distinguishing means may be disposed adjacent, above or below, but generally near the force-generating apparatus. The distinguishing means 950 may be an excitation laser which irradiates the components of the sample stream. Appropriate fluorescent tags attached to the components of the sample stream may allow the target particle of interest to fluoresce in response to the excitation laser. Laser fluorescence techniques may also be applied to other types of fluorescent chemistry, such as compounds which are expressed within the cells, rather than on the outside surface of the cell. Such compounds may include, for example, reagents which react with the human aldehyde dehydrogenase family of enzymes, and are available from Aldagen, Inc. of Durham, N.C.

The fluorescence signal may be detected by an optical system included in the distinguishing means 950. The optical system may include various lenses, optical filters and detectors as needed for the purpose. When a fluorescence event is detected, the detector may generate a signal which is monitored by the computer (not shown). The computer may then generate a trigger signal for the force-generating apparatus to generate the force to move the MEMS actuator in the MEMS cell sorting chip. The MEMS actuator may then direct the target particle of interest into the sort stream, and the remainder of the fluid into the waste stream. This operation may continue until one or more of the flexible bags 110 or 120 in the pressure chamber 100 is exhausted, or it is desired to process a new sample, or if the cell sorting system 1000 needs maintenance.

When any part of the removable/disposable apparatus 1 needs to be changed, for example due to clogging of the MEMS cell sorter chip 600, the entire removable/disposable apparatus 1 may be uninstalled from the cell sorting system 1000. This removable/disposable apparatus may include the components shown in FIG. 1. The pinch valves may first be activated, closing off the flow to the MEMS cell sorter chip 600 in the chip assembly 500. The chip assembly 500 may then be detached from the force-generating apparatus 900, and reattached to the filter assembly unit 400. Then, the pressure-generating apparatus may be disabled and the pressure chamber 100 is vented to atmosphere. A door to the pressure chamber 100 may then be opened and the bung 200 removed from the wall of the pressure chamber 100. The pinch valves 300 may be re-opened, the tubes 470 and 480 freed, and the sample and buffer bags then removed from the pressure chamber. The sort and waste receptacles 700 and 800 may be removed from the cell sorter system 1000. The chip assembly 500 may be detached from the predefined location adjacent to the force-generating apparatus 900 and clipped to the filter carrier 400 for removal. The filter carrier 400 with the chip assembly 500 may then be detached from the MEMS cell sorter system 1000 and any or all components of the removable/disposable apparatus 1 may be discarded or replaced.

Each of the reusable components of the removable/disposable apparatus are designed to be able to withstand the process which may be required to sterilize these components. Such processes may include heat, radiation, and physical or chemical cleaning treatment, such as autoclaving, ultrasound or air pulsing. Such sterilization procedures may be applied to any component which comes into contact with the sample fluid. The materials for the reusable components of the removable/disposable apparatus may be chosen to be amenable to the sterilization procedure intended to be performed on these components. However, since many of the components are intended to be disposed of between samples, they may be procured and assembled in a sterile condition. The materials used for these disposable components may include, as previously mentioned, PEEK for the tubing, PES for the filter, tygon or surgical tubing for the larger gauge tubes. Standard barbed polypropylene reducers may be used to adjust between different diameters of tubing. PES tubing may be used under the strain relief manifold 540. The MEMS cell sorting chips 600, and the flexible tubing 210 may not be not sterile upon assembly of the removable/disposable apparatus 1, but may be subjected to gamma irradiation or thermal treatments to achieve the necessary level of sterility. Bags and filters may be purchased in sterile condition.

A number of variations of the basic design concepts illustrated in FIGS. 1-6 may be envisioned. Although the bung 200 is a relatively inexpensive part, it may be reused rather than discarded, as it does not come into direct contact with the sample or buffer fluids. While in one embodiment, the MEMS cell sorter chip 600 is described as permanently mounted to the chip assembly 500, such that the entire chip assembly may be discarded, it may also be feasible and cost-effective to simply rework the chip assembly if the MEMS cell sorter chip becomes clogged. In this scenario, the old MEMS cell sorter chip is simply replaced with a new MEMS cell sorter chip 600. Furthermore, if the filter 410 becomes clogged, the filter 410 may be removed from the filter carrier 400 and replaced with a new filter 410 and the filter carrier 400 reused.

In any event, all of the components which come into direct contact with the sample may be removed and discarded relatively inexpensively. It should be understood that may of the dimensions and materials described above with respect to the components of the removable/disposable apparatus 1 are intended to be exemplary only.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. While the embodiment described above relates to a microelectromechanical human hematopoietic stem cell sorter, it should be understood that the techniques and designs described above may be applied to any of a number of other particle sorting applications. Other distinguishing means and/or force-generating means may be envisioned for such a particle sorting system, other than those described in the incorporated '056, '594 and '838 patents. Other types of MEMS particle sorting chips, such as those containing n×m arrays of microelectromechanical actuators and parallel channels, as well as one-dimensional 1×m arrays of such microelectromechanical actuators and parallel channel are contemplated according to the systems and methods described here. Furthermore, details related to the specific design features of the removable/disposable apparatus are intended to be illustrative only, and the invention is not limited to such embodiments. Finally, the systems and methods described herein may be used with non-mechanical particle sorting devices, such as microfluidic devices which use differential pressure, electric or magnetic fields to separate particles suspended in a fluid. Accordingly, the exemplary implementations set forth above, are intended to be illustrative, not limiting.

What is claimed is:

1. An apparatus for a particle sorting system, comprising:
a sample holder which holds at least one component of a sample stream;
a filter which receives the sample stream from the sample holder and filters particles from the sample stream, and is configured to be repeatably coupled to and decoupled from the particle sorting system;
first flexible tubing which delivers the sample stream from the sample holder to the filter carrier;
a compressible device disposed in or around the first flexible tubing; and
a chip assembly holding a microfabricated particle sorting chip which receives the sample stream from the filter.

2. The apparatus of claim 1, further comprising:
a filter carrier which holds the filter, the filter configured to remove particles larger than about 100 μm from the sample stream, and wherein the filter carrier is configured to be repeatably coupled to and decoupled from the particle sorting system.

3. The apparatus of claim 2, wherein the sample holder is a flexible bag, and wherein the apparatus comprises at least one additional flexible bag holding at least one additional component of the sample stream, wherein the at least one component is combined with the at least one additional component by a Y-connector disposed on the filter carrier between the flexible bags and the filter.

4. The apparatus of claim 3, further comprising:
second flexible tubing which delivers the sample stream from the filter carrier to the microfabricated particle sorting chip held in the chip assembly; and
a mechanism which delivers at least one of a mechanical and an acoustic disturbance to at least one of the first flexible tubing, the second flexible tubing, the filter and the microfabricated cell sorting chip.

5. The apparatus of claim 4, further comprising:
a sort receptacle which stores the component of interest separated from a remainder of the sample stream; and
a waste receptacle which stores the remainder of the sample stream.

6. The apparatus of claim 5, further comprising:
third flexible tubing which delivers the component of interest from the microfabricated particle sorting chip to the sort receptacle; and
fourth flexible tubing which delivers the remainder of the sample stream to the waste receptacle.

7. The apparatus of claim 6, wherein the compressible device is a rubber bung having a durometer of between about 20 and about 60 on a durometer A scale.

8. The apparatus of claim 1, wherein the microfabricated particle sorting chip comprises a MEMS actuator that can be moved by the application of lines of magnetic flux to the MEMS actuator.

9. The apparatus of claim 1, wherein the chip assembly is configured to be repeatably coupled to and decoupled from the particle sorting system, and wherein the microfabricated particle sorting chip is configured to separate the component from a remainder of the sample stream by interacting with a force-generating apparatus in the particle sorting system, wherein the force-generating apparatus is separable from the microfabricated particle sorting chip.

10. A particle sorting system, comprising:
the apparatus of claim 7; and
a pressure chamber which exerts pressure on at least one of the flexible bags causing the sample stream to flow from the at least one flexible bag, wherein the pressure in the pressure chamber is maintained by the deformable bung disposed in a wall of the pressure chamber.

11. The particle sorting system of claim 10, further comprising:
at least one valve which is adapted to stop a flow of the sample stream from at least one of the flexible bags; and
a disturbance device that generates at least one of a mechanical and an acoustic disturbance which is delivered to at least one of the first and second flexible tubing, the filter and the microfabricated particle sorting device.

12. The particle sorting system of claim 10, further comprising:
a distinguishing means that distinguishes a target particle from the remainder of the sample stream; and
a force-generating apparatus which causes a movement of a MEMS actuator in the microfabricated particle sorting chip, in response to s signal from the distinguishing means, but wherein the force-generating apparatus is separable from the microfabricated particle sorting chip.

13. The particle sorting system of claim 12, wherein the force-generating apparatus comprises an electromagnetic apparatus and wherein the distinguishing means is a laser and an optical detector.

14. A method for fabrication of a disposable apparatus, comprising:
coupling at least one sample holder to a filter carrier using a first flexible tubing, wherein the filter carrier is configured to be repeatably coupled to and decoupled from a particle sorting system;
disposing a compressible device in or around the first flexible tubing;
coupling the filter carrier to a microfabricated particle sorting chip using a second flexible tubing; and
coupling the microfabricated particle sorting chip to a sort receptacle using a third flexible tubing and to a waste receptacle using a a fourth flexible tubing.

15. The method of claim 12, further comprising:
installing the microfabricated particle sorting chip in a chip assembly with a manifold which holds the second, the third and the fourth flexible tubing in a stable position; and
detachably attaching the chip assembly to the filter carrier, such that the chip assembly is later detachable from the filter carrier upon installation in a particle sorting system.

16. A method for installing the apparatus of claim 6 in a particle sorting system, comprising:
coupling the filter carrier to a chassis of the particle sorting system;
placing the flexible bag within a pressure chamber; and
disposing the compressible device in a wall of the pressure chamber; and
detachably attaching the chip assembly to a predefined location relative to the particle sorting system.

17. A method for uninstalling the apparatus of claim 6 from a particle sorting system, comprising:
venting a pressure chamber to atmosphere;
opening a door to the pressure chamber;
removing the compressible device from a wall of the pressure chamber; and
removing the at least one flexible bag from the pressure chamber.

18. The method of claim 17, further comprising:
decoupling the chip assembly from the predefined location relative to the particle sorting system; and
coupling the chip assembly to the filter carrier.

19. The method of claim 18, further comprising:
decoupling the microfabricated particle sorting chip from the chip assembly;
coupling another microfabricated particle sorting chip to the chip assembly; and
reinstalling the chip assembly, filter carrier and at least one flexible bag in the particle sorting system.

20. The method of claim 17, further comprising:
decoupling the filter from the filter carrier;
coupling a new filter to the filter carrier;
coupling the new filter to the first and the second flexible tubing;
reinstalling the chip assembly, filter carrier and at least one flexible bag in the particle sorting system.
